(19) United States
(12) Patent Application Publication
(10) Pub. No.: US 2007/0286827 A1
(43) Pub. Date: Dec. 13, 2007

(54) OPTICAL BRIGHTER AND UV STABILIZER FOR ACRYLIC ENHANCEMENTS

(75) Inventors: Susan C. Sheariss, Swedesboro, NJ (US); Edward Sobolewski, Glen Mills, PA (US); Phon Malone, Turnersville, NJ (US); Anthony Calise, Aston, PA (US)

Correspondence Address: Connolly Bove Lodge & Hutz LLP;1007 North Orange Street, P.O. Box 2207, Wilmington, DE, 19899, US

(73) Assignee: ESSCHEM, INC., Linwood, PA (US)

(21) Appl. No.: 11/761,601

(22) Filed: Jun. 12, 2007

Related U.S. Application Data
(60) Provisional application No. 60/812,858, filed on Jun. 12, 2006.

(51) Int. Cl. A61K 8/81

(52) U.S. Cl. 424/61; 424/70.16

(57) ABSTRACT

An artificial nail composition containing multicarbonyl-vinyl containing monomer, polymerization initiator, and optical brightener. The invention also relates to the process of making the nail composition.

### RELATED APPLICATION

This application claims benefit U.S. Provisional application 60/812,858 filed Jun. 12, 2006 which is incorporated by reference in its entirety for all useful purposes.

### FIELD OF THE INVENTION

The invention is in the field of compositions for application to nails, which polymerize on the nail surface to yield an artificial nail structure. The resulting polymerized artificial nail will have improved optical brightness and improved UV stability for making artificial nails.

### BACKGROUND OF THE INVENTION

Artificial nails are widely used by women who desire to have long, attractive fingernails which do not break or chip as readily as natural nails. Artificial nails are generally formed directly on the natural nail surface by coating on the nail a layer of a liquid monomer and polymer. The material is shaped to the desired configuration, then the material is allowed to polymerize on the nail surface to yield a hard coating. The resulting artificial nail is then shaped and polished to look like a natural nail.

The preparation and application of artificial nails requires a certain level of skill. In addition, the monomer composition must exhibit certain properties, which enable easy application and polymerization of the composition on the nails, while at the same time providing a polymerized nail structure, which is strong, yet flexible. Important properties of the polymer are catalyst levels, particle size and, flexibility. Once the polymer and monomer composition is applied to the nails it must set, or polymerize, in a period of time, which is adequate to permit a skilled nail technician to apply and shape the composition. The hardened nail structure is flexible and strong.

Artificial nails have been known for years and are described in U.S. patent Publication 20060057082 (“the '082 publication”) and the background section of the patent publication. The '082 publication is incorporated by reference in its entirety for all usefull purposes.

There is a need to improve clarity and color brightness along with UV stabilization of the polymerized nail enhancement.

### SUMMARY OF THE INVENTION

It is an object of the invention to provide an artificial nail polymer composition, which exhibits improved clarity and color brightness, and improved UV stability.

It is a further object of the invention to provide a polymerized artificial nail structure that exhibits improved color brightness and UV stability.

It has been found that the above stated objectives can be realized in a liquid, artificial nail polymer composition comprised of one or more other addition-polymerizable, ethylenically unsaturated monomers, including in the composition a monoethylenically unsaturated vinyl monomer that contains two or more carbonyl groups (hereinafter referred to as a “multicarbonyl vinyl-containing monomer”), and in addition, including in the composition an optical brightener. Further, these objectives can be realized when incorporating this artificial nail polymer composition into an artificial nail structure.

The invention comprises a method for improving, optimizing, or enhancing the clarity and color brightness of a nail enhancement structure obtained by polymerizing on the natural nail surface a polymerizable monomer composition, during which polymerization process an effective amount of at least one multicarbonyl-vinyl containing monomer and optical brightener was added to said polymerizable monomer composition. The invention also comprises a method for improving, optimizing, or enhancing the clarity and color brightness of the artificial nail composition by adding an effective amount of optical brightener to said monomer during the polymerization process.

### DETAILED DESCRIPTION

All parts and percentages mentioned herein are parts and percentages by weight unless otherwise indicated.

The term “nail surface” means the natural nail surface, or a natural nail to which a pre-formed artificial nail or nail tip is adhered. In other words, the polymerizable compositions of the invention may be applied directly to the keratinous surface of the natural nail, or to a nail surface having affixed thereto a pre-formed artificial nail or nail tip enhancement.

The term “polymerizable” means that the composition polymerizes on the nail surface upon exposure to appropriate chemicals, heat, light (such as visibile or ultraviolet light), or other types of stimuli. The polymerization may occur by free radical or cationic mechanisms.

The Polymerizable Composition

The invention comprises a polymerizable monomer composition for application to the nails with an optical brightener, and polymerization thereon to yield an artificial nail structure. The polymerizable composition is preferably an anhydrous liquid, having the consistency of a semi-mobile gel to freely mobile liquid at room temperature. Immediately prior to use, the polymerizable composition is applied to the nail surface and shaped by the nail technician. After polymerization an artificial nail structure is obtained, which is then shaped and polished. The polymerizable composition of the invention contains at least one multicarbonyl-vinyl containing monomer and optical brightener. The polymerization composition may also contain a UV stabilizer.

The Multicarbonyl-Vinyl Containing Monomer

The composition preferably contains at least one multicarbonyl-vinyl containing monomer, preferably in the range of about 0.1-98.5%, more preferably 0.5-95%, most preferably 1-15%. The multicarbonyl-vinyl containing monomer includes those having the following general formula:

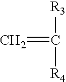

Wherein R₃ is H, a C₁₋₃₀ straight or branched chain alkyl, aryl, aralkyl; and R₄ is

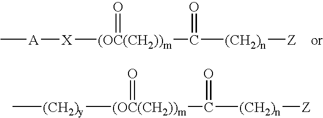

wherein

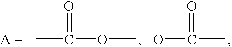

X=C₁₋₃₀ straight or branched chain alkyl, m is 1 to 5, n is 1 to 30, y is 0 to 50; and Z=H or a C₁₋₃₀ straight or branched chain alkyl.

Preferably R₃ is hydrogen or a C₁₋₈ alkyl, preferably methyl, and R₄ is

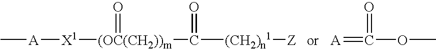

X¹=C₁₋₅ alkylene

m=1-5,

n¹=1-5, and

Z=C₁₋₁₀ straight chain alkyl.

Most preferably R₃ is hydrogen or methyl, and R₄ is as defined above wherein:

X=CH₂CH₂

m=1

n=1

Z=CH₃

In the preferred embodiment of the invention the multicarbonyl-vinyl containing monomer is a methacrylate, such as acetoacetoxyethyl methacrylate, which has the formula:

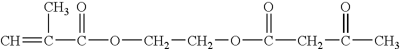

UV Stabilizer

Dicarboxylic acids including, but not limited to lactic, oxalic, malonic, glutaric, adipic, maleic, fumeric, tartaric, and phthalic acids are useful as UV stabilizers. Preferred carboxylic acid stabilizers are alpha-hydroxy acids. Preferred carboxylic acids are acetic, oxalic and lactic acids. In a preferred embodiment, the polymerizable compositions contain oxybenzone, also known as 2-hydroxy-4-methoxybenzophenone, (2-hydroxy-4-methoxyphenyl)phenylmethanone, and benzophenone-3, CAS #: 131-57-7, which has the formula:

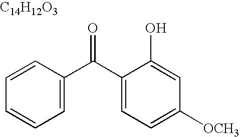

Polymerization Initiator/Accelerator

During the polymerization process, a polymerization initiator should be added which initiates or otherwise promotes the polymerization of the monomer. The initiator may be a thermal initiator, photo-initiator, or a chemical initiator. The Applicants do not limit their invention to any particular type of initiator.

Photo polymerization initiators or photo-initiators are known in the art and are described in U.S. Pat. No. 4,474,868. Preferred photo polymerization initiators for the purposes of this invention include UV initiators, gamma, and electron initiators.

Thermal initiators and chemical initiators are also known in the art. Common chemical initiators include free radical initiators and ion initiators such as cationic or anionic initiators. Preferred initiators for purposes of the present invention include peroxy initiators, barbituric acid, peroxide, or benzoyl acids. Most preferably, the initiator is benzyl peroxide. However, other polymerization promoting materials, such as free radical promoters and initiators, redox initiators, or metal catalysts may also be employed suitable to the chosen monomer and/or polymerizable composition.

Optical Brightener

An optical brightener should be added to the polymerizable composition in its liquid or suspension phase prior to solidification. Optical brighteners are known in the art, for example those described in U.S. Pat. No. 6,890,454. The Applicants do not limit their invention to any particular type of optical brightener. In a preferred embodiment of the present invention, the optical brightener is bis(t-butyl benzoxazolyl)thiophene. Other examples of optical brighteners which may be used in the present invention include 2,5-thiophenediylbis(5-tert-butyl-1-3-benzoxazole) and 4,4-bis(2-benzoxazolyl)stilbenzene.

Other Ingredients

The polymerizable compositions may additionally contain a variety of other ingredients, such as another ethylenically unsaturated monomer, a polymerization accelerator, plasticizer, U.V. absorber, polymerization regulator, pigments and/or polymer powder. Pigments include organic and inorganic pigments. Other useful pigments are pearlants such as mica, bismuth oxychloride and treated micas, such as titanated micas and lecithin modified micas. Some of the above are described in detail in U.S. Pat. Nos. 6,818,207 and 6,313,181.

The organic pigments include natural colorants and synthetic monomeric and polymeric colorants. Exemplary are phthalocyanine blue and green pigment, diarylide yellow and orange pigments, and azo-type red and yellow pigments such as toluidine red, litho red, naphthol red and brown pigments. Also useful are lakes, which are pigments formed by the precipitation and absorption of organic dyes on an insoluble base, such as alumina, barium, or calcium hydrates. Polymeric colorants include nylon powder, polyethylene, and polyesters. The polyesters can include linear, thermoplastic, crystalline or amorphous materials produced using one or more diols and one or more dicarboxylic acids copolymerized with colorants. An exemplary list of cosmetically acceptable colorants can be found in the International Cosmetic Ingredient Dictionary and Handbook, 7th Edition, CTFA, 1997, pp. 1628-1630, the contents of which are incorporated herein by reference. In the color cosmetics of the present invention, colorants other than the fluorescent brightener will normally constitute from about 0.1% to about 30% by weight of the composition, the amounts varying depending upon the color desired.

The inorganic pigments can include iron oxides (yellow, red, brown or black), ferric ammonium ferrocyanide (blue), manganese violet, ultramarine blue, chrome oxide (green), talc, lecithin modified talc, zeolite, kaolin, lecithin modified kaolin, titanium dioxide (white), and combinations thereof.

In addition, the polymerization composition may additionally contain a variety of antioxidants for protection against UV exposure. Antioxidants are described in Kirk-Othmers (vol. 3, pg. 424) and in Uhlmanns Encyclopedia (vol. 3, pg. 91) and several types of antioxidants suitable for this purpose are described in detail in U.S. Pat. No. 7,038,046. The Applicants do not limit their invention to any particular type of antioxidant.

Molding Techniques

The polymerizable compositions of the current invention may be used in extrusion molding processes, and may also be used in injection molding. Extrusion molding techniques are well known in the art. Extrusion molding involves extruding a molten polymer through a die to create long objects of a fixed cross-section. The injection molding of molten thermoplastic polymer is also well-known in the art. The conventional method of injection molding is to first inject the polymer melt into the closed mold and, subsequently, to pack additional melt into the cavity to compensate for the densification of the melt during the cooling stage.

The polymerizable compositions may also be used in blow molding, compression molding, thermoforming, and rotational molding. The current invention is not limited to a particular molding process.

Polymerization Process

The monomers described herein undergo addition polymerization generally at a temperature of about 50-125° C., and most preferably, at a temperature of about 70-100° C.

The Artificial Nail Structure

The invention is also directed to an artificial nail structure, having the physical properties disclosed in U.S. Pat. No. 6,818,207. The artificial nail structure contains optical brightener. Preferably the artificial nail structure comprises a copolymer of an ethylenically unsaturated monomer, and a multicarbonyl-vinyl containing monomer having the general formula set forth above. More preferably the artificial nail structure comprises a copolymer of one or more monofunctional ethylenically unsaturated monomers, one or more difunctional ethylenically unsaturated monomers, and one or more tri- or polyfunctional ethylenically unsaturated monomers and the di-carbonyl vinyl containing monomer. Most preferably, the artificial nail structure comprises a methacrylate copolymer, such as ethyl methacrylate, methyl methacrylate, hydroxpropyl methacrylate, acetoacetoxyethyl methacrylate, tetraethylene glycol dimethacrylate, or trimethylolpropane trimethacrylate ester. The artificial nail structure may also comprise a UV stabilizer. The artificial nail structure of the invention may also be made in a mold, and attached to the fingernail by adhesive or other similar means.

A Method for Improving the Optical Properties of Nail Enhancements

The invention also comprises a method for improving, optimizing, or enhancing the clarity and color brightness of a nail enhancement structure obtained by polymerizing on the natural nail surface a polymerizable monomer composition, during which polymerization process an effective amount of at least one multicarbonyl-vinyl containing monomer is added to said polymerizable monomer composition. Preferably, the multicarbonyl-vinyl containing monomer is as mentioned herein, and the polymerizable monomer composition comprises at least one ethylenically unsaturated monomer, preferably acetoacetoxyetyl methacrylate. An optical brightener is added during the polymerization process. In a preferred embodiment, bis(t-butyl benzoxazolyl)thiophene is the optical brightener added.

The amount of optical brightener which will improve clarity and color brightness ranges from about 0.001%-0.5%. The greater the amount of optical brightener, the greater the clarity and color brightness imparted.

A Method for Improving the UV Stability of Nail Enhancements

The invention also comprises a method for improving, optimizing, or enhancing the UV stability of a nail enhancement structure obtained by polymerizing on the natural nail surface a polymerizable monomer composition, during which polymerization process an effective amount of at least one multicarbonyl-vinyl containing monomer is added to said polymerizable monomer composition. Preferably, the multicarbonyl-vinyl containing monomer is as mentioned herein, and the polymerizable monomer composition comprises at least one ethylenically unsaturated monomer, preferably acetoacetoxyethyl methacrylate. UV stabilizer is also added during the polymerization process. In a prederred embodiment, oxybenzone is the UV stabilizer added.

The amount of UV stabilizer which will improve UV stability ranges from about 0.001%-1%. The greater the amount of UV stabilizer, the greater the UV stability imparted.

The invention will be further described in connection with the following examples, which are set forth for the purposes of illustration only.

### EXAMPLE 1

An artificial nail composition was made as follows:

| | w.w % |
|---|---|
| Ethyl methacrylate (EMA) | 70% (monomer) |
| Methyl methacrylate (MMA) | 30% (monomer) |
| Benzyl peroxide (BPO) | 1.10% (polymerization |
| | initiator/accelerator) |
| Bis(t-butyl benzoxazolyl) thiophene | 0.0059% (optical brighter) |

### EXAMPLE 2

An artificial nail composition was made as follows:

| | w.w % |
|---|---|
| Ethyl methacrylate (EMA) | 70% (monomer) |
| Methyl methacrylate (MMA) | 30% (monomer) |
| Benzyl peroxide (BPO) | 1.18% (polymerization |
| | initiator/accelerator) |
| Bis(t-butyl benzoxazolyl) thiophene | 0.0022% (optical brighter) |

### EXAMPLE 3

An artificial nail composition was made as follows:

| | w.w % |
|---|---|
| Ethyl methacrylate (EMA) | 70% (monomer) |
| Methyl methacrylate (MMA) | 30% (monomer) |
| Benzyl peroxide (BPO) | 1.15% (polymerization |
| | initiator/accelerator) |
| Bis(t-butyl benzoxazolyl) thiophene | 0.0154% (optical brighter) |

### EXAMPLE 4

An artificial nail composition was made as follows:

| | w.w % |
|---|---|
| Ethyl methacrylate (EMA) | 70% (monomer) |
| Methyl methacrylate (MMA) | 30% (monomer) |
| Benzyl peroxide (BPO) | 0.82% (polymerization |
| | initiator/accelerator) |
| Bis(t-butyl benzoxazolyl) thiophene | 0.033% (optical brighter) |
| Pigments | 2.938% |

### EXAMPLE 5

An artificial nail composition was made as follows:

| | w.w % |
|---|---|
| Ethyl methacrylate (EMA) | 70% (monomer) |
| Methyl methacrylate (MMA) | 30% (monomer) |
| Benzyl peroxide (BPO) | 1.44% (polymerization |
| | initiator/accelerator) |
| Bis(t-butyl benzoxazolyl) thiophene | 0.225% (optical brighter) |
| Pigments | 3.656% |

### EXAMPLE 6

An artificial nail composition was made as follows:

| | w.w % |
|---|---|
| Ethyl methacrylate (EMA) | 100% (monomer) |
| Benzyl peroxide (BPO) | 1.38% (polymerization |
| | initiator/accelerator) |
| Bis(t-butyl beuzoxazolyl) thiophene | 0.033% (optical brighter) |
| Pigments | 2.938% |

### EXAMPLE 7

An artificial nail composition was made as follows:

| | w.w % |
|---|---|
| Ethyl methacrylate (EMA) | 70% (monomer) |
| Methyl methacrylate (MMA) | 30% (monomer) |
| Benzyl peroxide (BPO) | 1.04% (polymerization |
| | initiator/accelerator) |
| Bis(t-butyl benzoxazolyl) thiophene | 0.0771% (optical brighter) |
| Benzophenone | 0.2467% (UV stabilizer) |
| Pigments | 3.304% |

### EXAMPLE 8

An artificial nail composition was made as follows:

| | w.w % |
|---|---|
| Methyl methacrylate (MMA) | 100% (monomer) |
| Benzyl peroxide (BPO) | 0.29% (polymerization |
| | initiator/accelerator) |
| Benzophenone | 0.2709% (UV stabilizer) |

### EXAMPLE 9

An artificial nail composition was made as follows:

| | w.w % |
|---|---|
| Ethyl methacrylate (EMA) | 70% (monomer) |
| Methyl methacrylate (MMA) | 30% (monomer) |
| Benzyl peroxide (BPO) | 0.98% (polymerization |
| | initiator/accelerator) |
| Bis(t-butyl benzoxazolyl) thiophene | 0.0257% (optical brighter) |
| Benzophenone | 0.2273% (UV stabilizer) |

### EXAMPLE 10

An artificial nail composition was made as follows:

| | w.w % |
|---|---|
| Ethyl methacrylate (EMA) | 65% (monomer) |
| Methyl methacrylate (MMA) | 35% (monomer) |
| Benzyl peroxide (BPO) | 1.13% (polymerization |
| | initiator/accelerator) |
| Bis(t-butyl benzoxazolyl) thiophene | 0.0264% (optical brighter) |
| Benzophenone | 0.2332% (UV stabilizer) |

### EXAMPLE 11

An artificial nail composition was made as follows:

| | w.w % |
|---|---|
| Ethyl methacrylate (EMA) | 70% (monomer) |
| Methyl methacrylate (MMA) | 30% (monomer) |
| Benzyl peroxide (BPO) | 1.10% (polymerization |
| | initiator/accelerator) |
| Bis(t-butyl benzoxazolyl) thiophene | 0.0059% (optical brighter) |
| Pigments | 0.1954% |

### EXAMPLE 12

An artificial nail composition was made as follows:

| | w.w % |
|---|---|
| Ethyl methacrylate (EMA) | 70% (monomer) |
| Methyl methacrylate (MMA) | 30% (monomer) |
| Benzyl peroxide (BPO) | 1.10% (polymerization |
| | initiator/accelerator) |
| Bis(t-butyl benzoxazolyl) thiophene | 0.0059% (optical brighter) |
| Pigments | 0.0461% |

### EXAMPLE 13

An artificial nail composition was made as follows:

| | w.w % |
|---|---|
| Ethyl methacrylate (EMA) | 70% (monomer) |
| Methyl methacrylate (MMA) | 30% (monomer) |
| Benzyl peroxide (BPO) | 1.10% (polymerization |
| | initiator/accelerator) |
| Bis(t-butyl benzoxazolyl) thiophene | 0.0059% (optical brighter) |
| Pigments | 0.2607% |

### EXAMPLE 14

An artificial nail composition was made as follows:

| | w.w % |
|---|---|
| Ethyl methacrylate (EMA) | 70% (monomer) |
| Methyl methacrylate (MMA) | 30% (monomer) |
| Benzyl peroxide (BPO) | 1.10% (polymerization |
| | initiator/accelerator) |
| Bis(t-bulyl benzoxazolyl) thiophene | 0.0059% (optical brighter) |
| Pigments | 0.0374% |

All the references described above are incorporated by reference in its entirety for all useful purposes. While there is shown and described certain specific structures embodying the invention, it will be manifest to those skilled in the art that various modifications and rearrangements of the parts may be made without departing from the spirit and scope of the underlying inventive concept and that the same is not limit to the particular forms herein shown and described.

We claim:

1. An artificial nail composition comprising multicarbonyl-vinyl containing monomer, polymerization initiator, and optical brightener.

2. The artificial nail composition of claim 1 wherein said optical brightener is present in an amount from about 0.001% to about 0.5%.

3. The artificial nail composition of claim 1 wherein said polymerization initiator is present in an amount from about 0.5%-3% polymerization initiator.

4. The artificial nail composition of claim 1 further comprising less than about 10% pigment.

5. The artificial nail composition of claim 1 wherein the optical brightener is bis(t-butyl benzoxazolyl)thiophene.

6. The artificial nail composition of claim 1 wherein the monomer is ethyl methacrylate, methyl methacrylate, or acetoacetoxyethyl methacrylate.

7. The artificial nail composition of claim 1 comprising at least one ethylenically unsaturated monomer.

8. The artificial nail composition of claim 1 wherein the polymerization initiator is benzyl peroxide.

9. The artificial nail composition of claim 1 further comprising a UV stabilizer.

10. The artificial nail composition of claim 9 wherein the UV stabilizer is about 0.001%-1.0% by weight of the artificial nail composition.

11. A method of enhancing the clarity and color brightness of the artificial nail composition of claim 1 comprising adding an effective amount of optical brightener to said monomer during the polymerization process.

12. The method of claim 11 which further comprises adding a UV stabilizer to said monomer during the polymerization process.

13. The method of claim 12 wherein the UV stabilizer is oxybenzone.

14. The method of claim 11 wherein the monomer is ethyl methacrylate, methyl methacrylate, or acetoacetoxyethyl methacrylate.

15. The method of claim 11 wherein the artificial nail composition comprises at least one ethylenically unsaturated monomer.

16. The method of claim 11 wherein the optical brightener is bis(t-butyl benzoxazolyl)thiophene.

17. The method of claim 12 wherein the UV stabilizer is about 0.001%-1.0% of the artificial nail composition.

18. The method of claim 11 wherein the optical brightener is about 0.001%-0.5% of the artificial nail composition.

19. The method of claim 11 wherein the artificial nail composition comprises about 0.5%-3% polymerization initiator.

20. The method of claim 11 wherein the polymerization initiator is benzyl peroxide.

21. A method of enhancing the UV stability of the artificial nail composition of claim 1 comprising adding an effective amount of UV stabilizer to said monomer during the polymerization process.

22. The method of claim 21 wherein the UV stabilizer is oxybenzone.

23. The method of claim 21 wherein the monomer is ethyl methacrylate, methyl methacrylate, or acetoacetoxyethyl methacrylate.

24. The method of claim 21 wherein the artificial nail composition comprises at least one ethylenically unsaturated monomer.

25. The method of claim 21 wherein the optical brightener is bis(t-butyl benzoxazolyl) thiophene.

26. The method of claim 21 wherein the UV stabilizer is about 0.001%-1.0% of the artificial nail composition.

27. The method of claim 21 wherein the optical brightener is about 0.001%-0.5% of the artificial nail composition.

28. The method of claim 21 wherein the artificial nail composition comprises about 0.5%-3% polymerization initiator.

29. The method of claim 21 wherein the polymerization initiator is benzyl peroxide.

30. An improved polymerized artificial nail enhancement composition having enhanced optical properties wherein the improvement comprises adding about 0.001%-0.5% optical brightener to the polymerizable nail composition during the polymerization process.
